(19) United States
(12) Patent Application Publication
(10) Pub. No.: US 2022/0195353 A1
(43) Pub. Date: Jun. 23, 2022

(54) DETERGENT AND/OR DISINFECTANT FOR WASHING AND/OR DISINFECTING

(71) Applicant: Diana GLElßNER, Tirschenreuth (DE)

(72) Inventor: Diana GLElßNER, Tirschenreuth (DE)

(21) Appl. No.: 17/338,289

(22) Filed: Jun. 3, 2021

(30) Foreign Application Priority Data
Dec. 22, 2020 (DE) DE102020134594.1

(51) Int. Cl. C11D 17/08; C11D 17/00

(52) U.S. Cl. CPC C11D 17/08; C11D 17/003

(57) ABSTRACT

Detergent and/or disinfectant for washing and/or disinfecting The present invention relates to a washing and/or disinfecting agent for washing and/or disinfecting surfaces, in particular skin surfaces, which comprises a base agent which is designed and intended for washing and/or disinfecting surfaces, in particular skin surfaces, with at least one substance additive being admixed to the base agent, which, after contact with the surface, assumes a different color consistency than the base agent for a predeterminable period of time, so that the substance additive serves as a marking means by means of which it is possible for the user to read whether the desired points of the surface to be washed and/or disinfected have been wetted with a sufficient amount of the base agent or have been wetted at all and/or a reaction time of the base agent has essentially been completed.

The present invention relates to a detergent and/or disinfectant for washing and/or disinfecting surfaces and to a use of a detergent and/or disinfectant as well as to a process for producing a detergent and/or disinfectant according to the respective generic term of claims 1, 6 and 7.

Particularly in the Corona pandemic times prevailing at the time of application, it is necessary to disinfect skin surfaces and/or other surfaces in areas where a high standard of hygiene is required, for example in a home for the elderly, a hospital or also towards weakened persons.

In this respect, the present invention relates to the task of specifying a washing and/or disinfecting agent for washing and/or disinfecting surfaces, in which it is possible in a particularly simple manner to determine whether a degree of action and/or sufficient surface wetting prevails.

This makes it possible to dispense with costly measurement procedures or even with a specific test procedure, which is based in particular on the empirical values of the respective user.

According to at least one embodiment, the washing and/or disinfecting agent for washing and/or disinfecting surfaces, in particular skin surfaces, comprises a base agent which is set up and intended for washing and/or disinfecting surfaces, in particular skin surfaces, at least one substance additive being admixed to this base agent, which, after contact with the surface, assumes a different color consistency than the base agent for a predeterminable period of time, so that the substance additive serves as a marking means by means of which it is possible for the user to read whether the desired areas of the surface to be washed and/or disinfected have been wetted with a sufficient amount of the base agent or have been wetted at all and/or a contact time of the base agent has been substantially completed.

According to at least one embodiment, the substance additive is admixed with at least 3 vol % and/or at least 3 mol % of the base agent and, in particular, a predetermined amount by weight and/or volume of the base agent.

According to at least one embodiment, the substance additive comprises at least one colorant which reflects and/or generates light in the visible wavelength spectrum.

According to at least one embodiment, the additive is added to the base composition in gel and/or viscous and/or liquid form

According to at least one embodiment, the additive comprises at least one time marker which, after application to the surface, in particular to the skin surface, based on the dye, glows on the surface for a predetermined period of time, and after this period of time has elapsed, the glowing at least partially stops, indicating to the user that an exposure time of the base agent has elapsed.

According to at least one embodiment, the present invention relates to a use of a washing and/or disinfecting agent.

According to at least one embodiment, the process for producing a washing and/or disinfecting agent for washing and/or disinfecting surfaces comprises a base agent which is set up and intended for washing and/or disinfecting surfaces, in particular skin surfaces, and the provision of a substance additive which, after contact with the surface, assumes a different color consistency from the base agent for a predeterminable period of time, so that the substance additive serves as a marking means by which the user can read off whether the desired points of the surface to be washed and/or disinfected have been wetted with a sufficient amount or at all with the base agent and/or an exposure time of the base agent has been substantially completed, this additive being admixed with the bathing agent.

In this context, the substance additive can be a luminescent and/or fluorescent agent or at least have such a luminescent and/or fluorescent agent. This may be a light spectrum visible to the human eye. In this context, “gel-like” means that the base agent has a consistency with essential general conditions of a gel. For example, the additive may actually be a gel.

The same applies accordingly to the terms viscous as well as liquid.

Namely, a viscous substance may be one which has a viscosity indicating that the higher the viscosity, the thicker (less flowable) a fluid is, and the lower the viscosity, the thinner (more flowable) it is.

For example, the additive is a viscous or gel-like substance.

It is conceivable that base forms a suspension together with the additive.

It is also conceivable that the additive forms an emulsion together with the base substance, or that the additive is dissolved in the base substance so that a solution develops.

The time marker described here can be a chemical element either in the additive and/or in the base substance, which changes after contact with ambient oxygen and/or after contact with a skin surface and therefore a corresponding chemical and/or physical process is triggered within the time marker, which takes place over a predetermined period of time. It is conceivable that after the distribution of the detergent and/or disinfectant on the skin surface, the user perceives a glow or other color coding for a period of 30 seconds or 40 seconds or any other period that can be set on the basis of the substance mixture and the time marker. This color coding disappears after a predetermined period of time, which is preferably measured in such a way that this indicates that a sufficient disinfection exposure time has been exceeded. In other words, if only the glow has disappeared in principle, sufficient disinfection has been achieved with regard to a reduction in bacteria and/or viruses or a corresponding complete destruction.

In the following, the invention described herein is illustrated in more detail with reference to a FIGURE and the associated description.

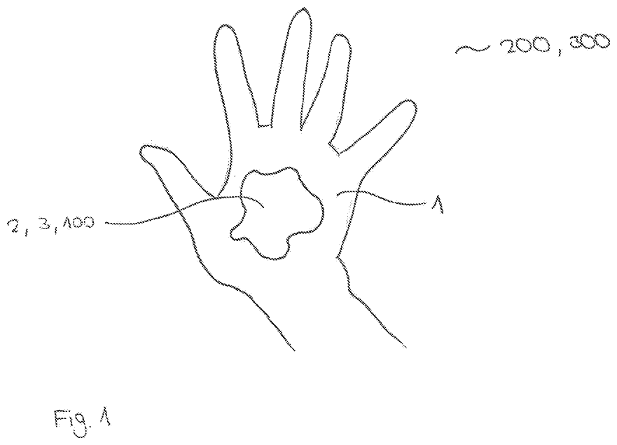

In FIG. 1, it can be seen in a top view of a surface 1, in particular a skin surface 1, that the washing and/or disinfecting agent 100 used there is for washing and/or disinfecting surfaces 1, in particular skin surfaces 1.

A base agent is recognizable which is set up and intended for washing and/or disinfecting surfaces 1, in particular skin surfaces 1, with it, wherein at least one substance additive 3 is admixed to this base agent, which substance additive 3 assumes a different color consistency than the base agent 2 for a predeterminable period of time after contact with the surface 1, so that the substance additive 3 serves as a marking means by which the user can read off whether the desired points of the surface 1 to be washed and/or disinfected have been wetted with a sufficient amount or at all with the base agent and/or a contact time of the base agent has been substantially completed.

The substance additive 3 is admixed with at least 3% by volume and/or at least 3 mol % of the base composition and, in particular, a predetermined amount by weight and/or volume of the base composition and comprises at least one dye which reflects and/or generates light in the visible wavelength spectrum.

It can be seen that the substance additive 3 is added to the base agent in gel and/or liquid form.

This additive 3 comprises at least one time marker which, after application to the surface 1, in particular to the skin surface 1, lights up on the basis of the dye for a predetermined period of time on the surface 1, and after expiry of this period of time at least partially stops lighting up, indicating to the user that an exposure time of the base agent has expired.

FIG. 1 thus shows a washing and/or disinfecting agent for washing and/or disinfecting surfaces 1 and a use of a washing and/or disinfecting agent 200 as well as a method for producing a washing and/or disinfecting agent 300 for washing and/or disinfecting surfaces 1, which comprises a blowing agent which is adapted and provided for washing and/or disinfecting surfaces 1, in particular skin surfaces 1, therewith.

In addition, the process 300 comprises the provision of a substance additive 3, which is admixed with the base composition, which after contact with the surface 1 assumes a different color consistency than the base composition 2 for a predeterminable period of time, so that the substance additive 3 serves as a marking means, by means of which it can be read by the user whether the desired areas of the surface 1 to be washed and/or disinfected have been wetted with a sufficient amount of the base composition or have been wetted at all and/or a contact time of the base composition has been substantially completed.

### LIST OF REFERENCE SIGNS

1 Surface, skin surface
2 Basic means
3 Substance additive
100 Detergent and/or disinfectant
200 Use of a washing and/or disinfecting agent
300 Process of a washing and/or disinfecting agent

1. A washing and/or disinfecting agent (100) for washing and/or disinfecting surfaces (1), in particular skin surfaces (1), comprising
a base agent (2) which is adapted and intended for washing and/or disinfecting surfaces (1), in particular skin surfaces (1), therewith,
characterized in that
at least one substance additive (3) is admixed to the base agent (2), which substance additive (3), after contact with the surface (1), assumes a different color consistency than the base agent (2) for a predeterminable period of time, so that the substance additive (3) serves as a marking agent, by means of which it can be read by the user whether the desired points of the surface (1) to be washed and/or disinfected have been wetted with a sufficient amount or at all with the base agent (2) and/or a reaction time of the base agent (2) has been substantially completed.

2. The washing and/or disinfecting agent (100) according to claim 1,
characterized in that
the substance additive (3) is admixed with at least 3% by volume and/or at least 3 mol % of the base composition (2) and in particular of a predetermined amount by weight and/or volume of the base composition (2).

3. The washing and/or disinfecting agent (100) according to claim 1,
characterized in that
the substance additive (3) comprises at least one dye which reflects and/or generates light in the visible wavelength spectrum.

4. The washing and/or disinfecting agent (100) according to claim 1,
characterized in that
the substance additive (3) is added to the base composition (2) in gel and/or viscous and/or liquid form.

5. The washing and/or disinfecting agent (100) according to claim 1,
characterized in that
the additive (3) comprises at least one time marker which, after application to the surface (1), in particular to the skin surface (1), lights up on the surface (1) for a predetermined period of time on the basis of the colorant, and after expiry of this period of time the lighting up at least partially stops, indicating to the user that an exposure time of the base composition (2) has expired.

6. A use of a washing and/or disinfecting agent (200) according to claim 1 for washing and/or disinfecting surfaces (1), in particular skin surfaces (1).

7. A method of producing a washing and/or disinfecting agent (300) for washing and/or disinfecting surfaces (1) comprising the steps of:
providing a base agent (2) which is adapted and intended to be used for washing and/or disinfecting surfaces (1), in particular skin surfaces (1),
providing a substance additive (3) which, after contact with the surface (1), assumes a different color consistency than the base agent for a predeterminable period of time, so that the substance additive (3) serves as a marking means by means of which it can be read by the user whether the desired areas of the surface (1) to be washed and/or disinfected have been wetted with a sufficient amount of the base agent (2) or at all, and/or an action time of the base agent (2) has essentially been completed, characterized in that
the substance additive (3) is added to the base agent (2).
